(19) United States
(12) Patent Application Publication
(10) Pub. No.: US 2020/0015566 A1
(43) Pub. Date: Jan. 16, 2020

(54) SYSTEM FOR APPLYING A HAIR REMOVER TO HUMAN SKIN AND THE HAIR REMOVER

(71) Applicant: Rosana Michail, New York, NY (US)

(72) Inventor: Rosana Michail, New York, NY (US)

(21) Appl. No.: 16/443,988

(22) Filed: Jun. 18, 2019

Related U.S. Application Data
(60) Provisional application No. 62/763,995, filed on Jul. 12, 2018.

(51) Int. Cl. A45D 26/00; A61Q 9/04; A61K 8/92

(52) U.S. Cl. CPC A45D 26/0014; A61K 8/925; A61Q 9/04

(57) ABSTRACT

A system that applies a hair remover to human skin and the hair remover. The system includes a depilatory and an applicator. The depilatory removes hair from the human skin. The applicator applies the depilatory to the human skin. The depilatory is a combination of various all natural ingredients including bees wax, rosin, and natural plant-based oils. The applicator is a brush, rather than a wooden stick as a result of viscosity and properties of the combination of various all natural ingredients of the depilatory versus prior art depilatory waxes.

### CROSS REFERENCE TO RELATED APPLICATIONS

The instant non-provisional patent application claims priority from provisional patent application No. 62/763,995, filed Jul. 12, 2018, and incorporated herein in its entirety by reference thereto

### BACKGROUND OF THE INVENTION

### Field of the Invention

The embodiments of the present invention relate to a system, and more particularly, the embodiments of the present invention relate to a system for applying a hair remover to human skin and the hair remover.

### Description of the Prior Art

Epilation by application of a hot depilatory wax to the skin and removal of the wax from the skin after cooling is an accepted technique for effective and long-lasting hair removal. As the melted ax applied to the skin begins to harden a cloth is pressed thereagainst. After the wax hardens, the cloth is pulled back and pulls the wax and hair captured thereby from a human's pores. In the prior art, a wooden stick similar to a tongue depressor is used to apply the wax.

The accepted commercial technique of removing hair by application of a hot wax depilatory usually includes heating a large quantity of depilatory wax to a molten state in a large and open vat. The individual then tests the temperature of the wax by touching. Generally, a skilled individual will apply the melted wax by dipping a spoon or stick applicator into the vat and collecting a blob of molten wax on the applicator and coating the skin in the area where hair is to be removed.

Wax is wasted due to the dripping and spilling as the applicator is removed from the vat. Moreover, when the wax is heated to a molten state for application to the skin, it retains a high viscosity similar to that of molasses or thick honey due to the properties thereof. Because of this high viscosity property, the depilatory wax tends to be applied to the skin in thick blobs.

It is noted, however, that depilatory wax is like candle wax, and thus, when molten, can cause severe pain and burning of the skin if applied in thick blobs. Moreover, the imprecise application of the molten wax using a spoon or a stick does not permit the thickness of the layer of wax applied to be controlled to any great degree, and the thicker the wax layer applied, the less hair is removed by peeling of the wax from the skin after hardening.

Numerous innovations for depilatory-related systems have been provided in the prior art, which will be described, infra, in chronological order to show advancement in the art, and which are incorporated herein in their entirety by reference thereto. Even though these innovations may be suitable for the specific individual purposes to which they address, however, they differ from the embodiments of the present invention. For example:

### U.S. Pat. No. 4,773,784 to Mann

U.S. Pat. No. 4,773,784—issued to Mann on Sep. 27, 1988 in U.S. Class 401 and subclass 1—teaches a system for heating and applying a depilatory wax to skin for removal of hair. The system includes a reservoir that stores a supply of the wax and a dispenser coupled to the reservoir. The depilatory wax is essentially solid at room temperature, but becomes flowable when heated. The heated wax flows from the reservoir to the dispenser for application. The roller applies the wax in a thin layer to prevent pain and burning.

### U.S. Pat. No. 4,958,951 to Mann

U.S. Pat. No. 4,958,951—issued to Mann on Sep. 25, 1990 in U.S. Class 401 and subclass 1—teaches a system for heating and applying a depilatory wax to skin for removal of hair. The system includes a reservoir that stores a supply of the wax and a dispenser coupled to the reservoir. The depilatory wax is essentially solid at room temperature, but becomes flowable when heated. The heated wax flows from the reservoir to the dispenser for application. The roller applies the wax in a thin layer to prevent pain and burning.

### U.S. Pat. No. Des. 333,883 to Mann

U.S. Pat. No. Des. 333,883—issued to Mann on Mar. 9, 1993 in U.S. Class D28 and subclass 44.1—teaches the ornamental design for a hot wax hair remover apparatus.

### U.S. Pat. No. 5,847,363 to Debourg et al.

U.S. Pat. No. 5,847,363—issued to Debourg et al. on Dec. 8, 1998 in U.S. Class 219 and subclass 424—teaches a hair removal wax device including at least one tank linked to a heating apparatus, and a detachable instrument that is independent from the tank and designed for sampling from the tank a quantity of molten wax. An apparatus transfers a quantity of hair removal wax from the tank to the detachable instrument. The detachable instrument is a ductile vessel whose inside volume is variable.

### U.S. Pat. No. 6,039,482 to Smal

U.S. Pat. No. 6,039,482—issued to Smal on Mar. 21, 2000 in U.S. Class 401 and subclass 1—teaches a hot wax hair remover apparatus including a heating sleeve for depilatory wax equipped with a wax applicator. The heating sleeve is provided with a retractable foot equipped with a spring holding the foot in a deployed position and with apparatus for locking the foot in a retracted position, against the force of the spring and controlled by a catch arranged at the base of the sleeve that releases the locking apparatus when the sleeve is put down on a support surface.

### U.S. Pat. No. 6,478,493 to Cepeda et al.

U.S. Pat. No. 6,478,493—issued to Cepeda et al. on Nov. 12, 2002 in U.S Class 401 and subclass 49—teaches a depilatory wax stick that is for use, particularly, on facial and neck surfaces and other small areas and even in delicate areas where very fine wax deposition is required. In a preferred embodiment, the wax stick is generally cylindrical and is about 10 cm long and has a slightly tapered shape with a 1.8 cm diameter base and a 1.5 cm diameter free end that is rounded to form about a 1.0 cm application tip. Because there is no required flow of wax through an aperture or passage or onto a roller, the stick may be shaped in virtually unlimited ways to facilitate extremely fine application control. In its simplest form, the base of the stick is held in a tapered plastic cap that provides a stable flat surface. An external housing may be provided to promote convenient storage and to prevent contamination of the wax between uses. In an alternative embodiment, the wax stick is provided in a push-up container, much like a lipstick tube that may be capped to enclose the stick.

### United States Patent Application Publication Number 2004/0175340 to Gupta

United States Patent Application Publication Number 2004/0175340—published to Gupta on Sep. 9, 2004 in U.S. Class 424 and subclass 70.1—teaches cosmetic cold-wax hair removal (depilatory) compositions suitable for site-specific face or body applications, and having the attributes of a pre-heating or microwave step not being necessary, complete hair removal is achieved in a single application on a specific site or area, no (or minimal) skin irritation is experienced, bond to hair preferentially, and not to skin, provide lubricity and emolliency to skin, and can be applied either directly on the skin or, preferably, on a piece of precut fabric, plastic, or paper, which is then applied to a specific site where hair removal is desired. Alternatively, dispensing applicators can also be used that can be filled for delivery at the specific site where depilation is desired, and having the further attributes of requiring minimal clean-up with water or common make-up removal compositions, and additional skin and hair beneficial ingredients, such as, hair growth retardants, anti-irritants, topical pain relief agents, antioxidants, skin soothing agents, skin cooling agents, emollients, moisturizers, topical anesthetics, colorants, botanical extracts, and fragrances can be included.

### United States Patent Application Publication Number 2009/0163933 to DiMaio

United States Patent Application Publication Number 2009/0163933—published to DiMaio on Jun. 25, 2009 in U.S Class 606 and subclass 134—teaches an ear hair removal instrument including a head, at least a portion of which, i.e., its outer surface is covered with cold wax. The head is shaped and sized to be receivable in the outer ear, while preventing insertion into the middle ear. A handle is mounted, at its distal end, to the head.

In use, the head of the instrument is inserted in the outer ear so that the cold wax disposed on the head comes into contact with the ear hair. While disposed in the outer ear, the ear hair in contact with the cold wax is extracted from its follicles by simultaneously rotating and pulling the instrument.

United States Patent Application Publication Number 2012/0023682 to Radha

United States Patent Application Publication Number 2012/0023682—published to Radha on Feb. 2, 2012 in U.S Class 8 and subclass 161—teaches a natural alternative to wax to remove unwanted hair, which does not cause irritation to any type of skin. It is made of sugar, water, and lemon. It does not need a special machine to heat it, and does not take time to reach the right temperature to be ready for use. It is environmentally friendly because it does not need disposal wooden sticks to apply it, and does not need disposal sheets to remove unwanted hair. It also does not need lotions after removing the unwanted hair because it does not dry the skin, and keeps it smooth. It is also affordable that is enough to apply on full body many times. The hair does not grow after the application of the natural alternative at least for three to six weeks.

### United States Patent Application Publication Number 2016/0120288 to Keren et al.

United States Patent Application Publication Number 2016/0120288—published to Keren et al. on May 5, 2016 in U.S. Class 606 and subclass 134—teaches a hair removal device using, or for use in combination with, a wax-strip configured to remove hair from skin. The device includes a user holding-member having a housing or chassis with a wax-strip removal mechanism actuator. The housing has a bottom surface configured for engaging with the skin. The device further includes a wax-strip engagement mechanism for removable engagement with the wax-strip, a wax-strip removal mechanism having a wax-strip removal acceleration arrangement, and a wax-strip pressing mechanism. The wax-strip pressing mechanism presses the wax-strip to the user's skin by way of a pressing member and also distances the pressing member from the skin during operation of the device to help mitigate shear and/or rubbing on the skin.

### U.S. Pat. No. 9,913,521 to Keren et al.

U.S. Pat. No. 9,913,521—issued to Keren et al. on Mar. 13, 2018 in U.S. Class 1 and subclass 1—teaches a hair removal device using, or for use in combination with, a wax-strip configured to remove hair from skin. The device includes a user holding member having a housing or chassis with a wax-strip removal mechanism actuator. The housing has a bottom surface configured for engaging with the skin. The device further includes a wax-strip engagement mechanism for removable engagement with the wax-strip, a wax-strip removal mechanism having a wax-strip removal acceleration arrangement, and a wax-strip pressing mechanism. The wax-strip pressing mechanism presses the wax-strip to the user's skin by way of a pressing member and also distances the pressing member from the skin during operation of the device to help mitigate shear and/or rubbing on the skin.

It is apparent that numerous innovations for depilatory-related devices have been provided in the prior art that are adapted to be used. Furthermore, even though these innovations may be suitable for the specific individual purposes to which they address, however, they would not be suitable for the purposes of the embodiments of the present invention as heretofore described.

### SUMMARY OF THE INVENTION

Thus, an object of the embodiments of the present invention is to provide a system for applying a hair remover to human skin and the hair remover, which avoids the disadvantages of the prior art.

Briefly stated, another object of the embodiments of the present invention is to provide a system that applies a hair remover to human skin and the hair remover. The system includes a depilatory and an applicator. The depilatory removes hair from the human skin. The applicator applies the depilatory to the human skin. The depilatory is a combination of various all natural ingredients including bees wax, rosin, and natural plant-based oils. The applicator is a brush rather than a wooden stick as a result of viscosity and properties of the combination of various all natural ingredients of the depilatory versus prior art depilatory waxes.

The embodiments of the present invention themselves, however, both as to their construction and to their method of operation, together with additional objects and advantages thereof, will be best understood from the following description of the embodiments of the present invention when read and understood in connection with the accompanying figures of the drawing.

### BRIEF DESCRIPTION OF THE FIGURES OF THE DRAWING

The figures of the drawing are briefly described as follows:

FIG. 1 is a diagrammatic perspective view of the system of the embodiments of the present invention applying a hair remover to human skin and removing hair from the human skin;

FIG. 2 is a diagrammatic perspective view of the system of the embodiments of the present invention;

FIG. 3 is an exploded diagrammatic perspective view of the system of the embodiments of the present invention;

FIG. 4 is a diagrammatic front perspective view of the applicator of the system of the embodiments of the present invention identified by ARROW 4 in FIGS. 2 and 3;

FIG. 5 is a diagrammatic rear perspective view taken in the direction of ARROW 5 in FIG. 4 of the applicator of the system of the embodiments of the present invention; and

FIG. 6A-6B is a diagrammatic flowchart of the method of using the system of the embodiments of the present invention.

### LIST OF REFERENCE NUMERALS UTILIZED IN THE FIGURES OF THE DRAWING

### Introductory

10 system of embodiments of present invention for applying hair remover 12 to human skin 14 and hair remover 12
12 hair remover for removing hair 16 from human skin 14
14 human skin
16 hair

### Overall Configuration of System 10

18 depilatory for removing hair 16 from human skin 14
20 applicator for applying depilatory 18 to human skin 14

### Specific Configuration of Depilatory 18

22 vessel containing depilatory 18
24 rim of vessel 22 containing depilatory 18
26 combination of various all natural ingredients of depilatory 18
28 bees wax of combination of various all natural ingredients 26 of depilatory 18
30 rosin of combination of various all natural ingredients 26 of depilatory 18
32 natural plant-based oils of combination of various all natural ingredients 26 of depilatory 18

### Specific Configuration of Applicator 20

34 brush of applicator 20
36 handle of brush 34 of applicator 20
38 plurality of bristles of brush 34 of applicator 20
40 proximal end of handle 36 of brush 34 of applicator 20
42 distal end of handle 36 of brush 34 of applicator 20
44 midsection of handle 36 of brush 34 of applicator 20

### Method of Using the System 10

46 melted depilatory of depilatory 18
48 wax-laden bristles of brush 14 of applicator 20;
50 applied depilatory of depilatory 18
52 strip of cooled depilatory of depilatory 18

### DETAILED DESCRIPTION OF THE PREFERRED EMBODIMENTS

### Introductory

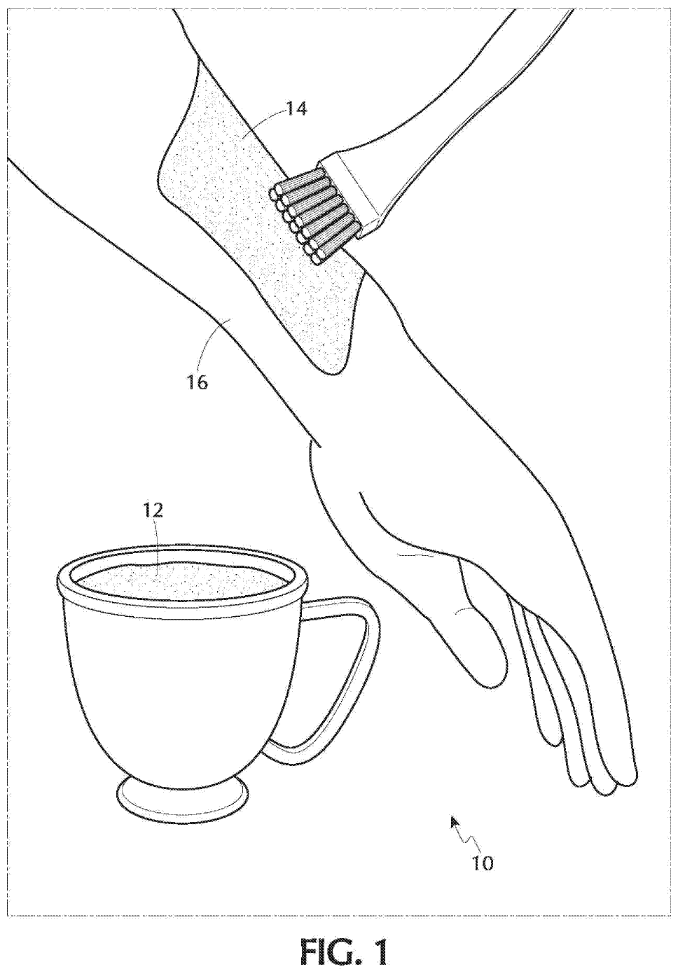

Referring now to the figures, in which like numerals indicate like parts, and particularly to FIG. 1, the system of the embodiments of the present invention is shown generally at 10 for applying a hair remover 12 to human skin 14.

### Overall Configuration of the System 10

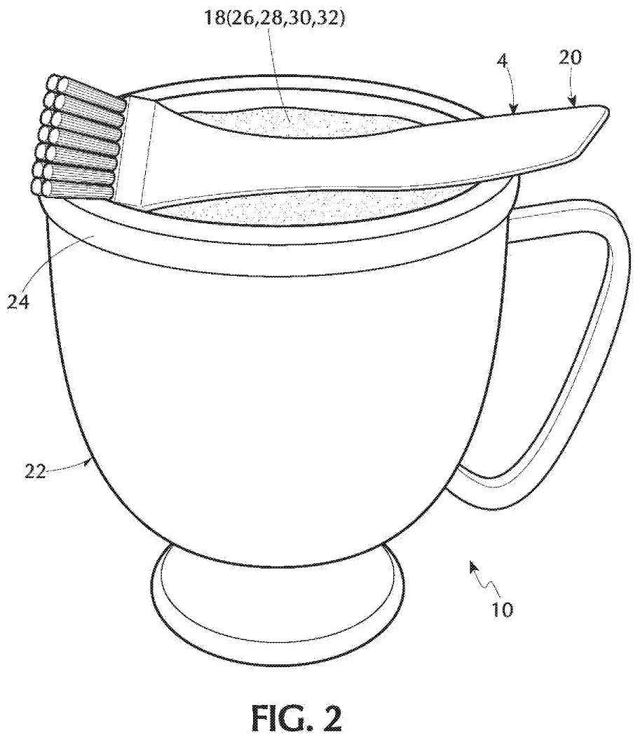

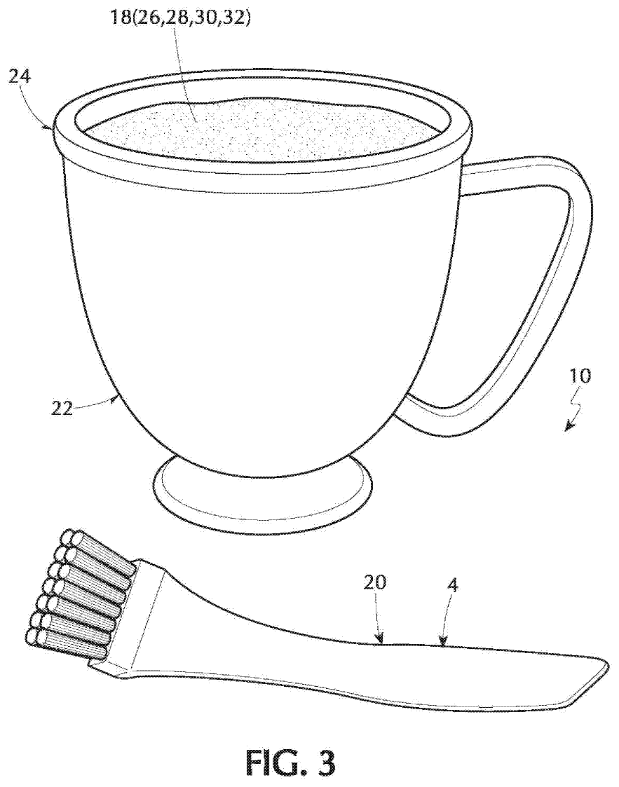

The overall configuration of the system 10 can best be seen in FIGS. 2 and 3, and as such, will be discussed with reference thereto.

The system 10 comprises a depilatory 18 and an applicator 20. The depilatory 18 is for removing hair 16 from the human skin 14 (FIG. 1). The applicator 20 is for applying the depilatory 18 to the human skin 14 (FIG. 1).

### Specific Configuration of the Depilatory 18

The specific configuration of the depilatory 18 can best be seen in FIGS. 2 and 3, and as such, will be discussed with reference thereto.

The depilatory 18 is contained in a vessel 22 having a rim 24.

The depilatory 18 is a combination of various all natural ingredients 26.

The combination of various all natural ingredients 26 of the depilatory 18 include bees wax 28, rosin 30, and natural plant-based oils 32.

### Specific Configuration of the Applicator 20

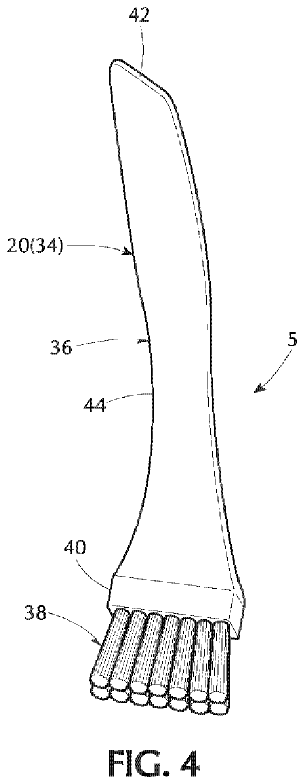

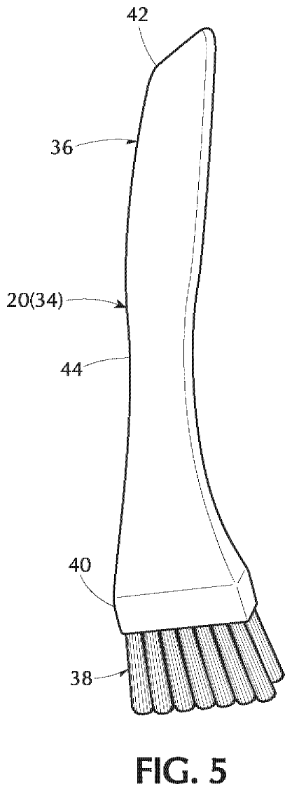

The specific configuration of the applicator 20 can best be seen in FIGS. 4 and 5, and as such, will be discussed with reference thereto.

The applicator 20 is a brush 34, rather than a wooden stick similar to a tongue depressor of the prior art, because of viscosity and properties of the combination of various all natural ingredients 26 of the depilatory 18 versus prior art depilatory waxes.

In the prior art, a wooden stick similar to a tongue depressor is used to apply wax, which is used in the prior art by other depilatory waxes.

The brush 34 of the applicator 20 has a handle 36 and a plurality of bristles 38.

The handle 36 of the brush 34 of the applicator 20 has a proximal end 40, a distal end 42, and a midsection 44 therebetween.

The plurality of bristles 38 of the brush 34 of the applicator 20 extend axially from the proximal end 38 of the handle 36 of the brush 34 of the applicator 20.

The midsection 44 of the handle 36 of the brush 34 of the applicator 20 is arcuate so as to allow the brush 34 of the applicator 20 to rest safely across the rim 24 of the vessel 22 between applications of the depilatory 18 (FIG. 2).

The handle 36 of the brush 34 of the applicator 20 is 5″ long and made of plastic, while the plurality of bristles 38 of the brush 34 are 1″ long and synthetic.

### Method of Using the System 10

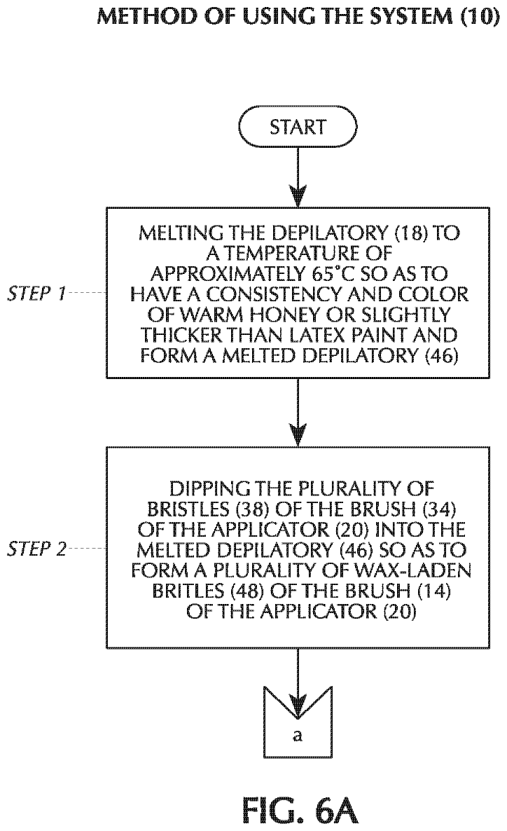

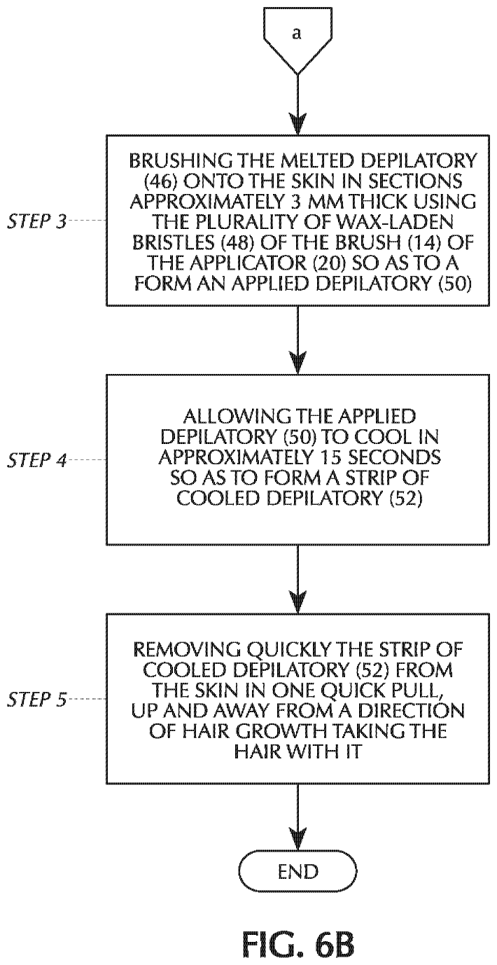

The method of using the system 10 can best be seen in FIGS. 1 and 6A-6B, and as such, will be discussed with reference thereto.
STEP 1: As shown in FIG. 6A, melting the depilatory 18 to a temperature of approximately 65° C. so as to have a consistency and color of warm honey or slightly thicker than latex paint and form a melted depilatory 46;
STEP 2: As further shown in FIG. 6A, dipping the plurality of bristles 38 of the brush 34 of the applicator 20 into the melted depilatory 46 so as to form a plurality of wax-laden bristles 48 of the brush 14 of the applicator 20;
STEP 3: As shown in FIG. 6B, brushing the melted depilatory 46 onto the skin in sections approximately 3 mm thick using the plurality of wax-laden bristles 48 of the brush 14 of the applicator 20 so as to a form an applied depilatory 50;
STEP 4: As further shown in FIG. 6B, allowing the applied depilatory 50 to cool in approximately 15 seconds so as to form a strip of cooled depilatory 52; and
STEP 5: As still further shown in FIG. 6B Removing quickly the strip of cooled depilatory 52 from the skin in one quick pull, up and away from a direction of hair growth taking the hair with it.

### Impressions

It will be understood that each of the elements described above, or two or more together, may also find a useful application in other types of constructions differing from the types described, supra.

While the embodiments of the present invention have been illustrated and described as embodied in a system for applying a hair remover to human skin and the hair remover, nevertheless, they are not limited to the details shown, since it will be understood that various omissions, modifications, substitutions, and changes in the forms and details of the embodiments of the present invention illustrated and their operation can be made by those skilled in the art without departing in any way from the spirit of the embodiments of the present invention.

Without further analysis, the foregoing will so fully reveal the gist of the embodiments of the present invention that others can, by applying current knowledge, readily adapt them for various applications without omitting features from the standpoint of prior art fairly constitute characteristics of the generic or specific aspects of the embodiments of the present invention.

The invention claimed is:

1. A system for applying a hair remover to human skin and the hair remover, comprising:
a) a depilatory; and
b) an applicator;
wherein said depilatory is for removing hair from the human skin; and
wherein said applicator is for applying said depilatory to the human skin.

2. The system of claim 1, wherein said depilatory is contained in a vessel.

3. The system of claim 2, wherein said vessel containing said depilatory has a rim.

4. The system of claim 1, wherein said depilatory is a combination of various all natural ingredients.

5. The system of claim 4, wherein said combination of various all natural ingredients of said depilatory include bees wax.

6. The system of claim 4, wherein said combination of various all natural ingredients of said depilatory include rosin.

7. The system of claim 4, wherein said combination of various all natural ingredients of said depilatory include natural plant-based oils.

8. The system of claim 4, wherein said combination of various all natural ingredients of said depilatory include bees wax, rosin, and natural plant-based oils.

9. The system of claim 3, wherein said applicator is a brush rather than a wooden stick as a result of viscosity and properties of said combination of various all natural ingredients of said depilatory versus prior art depilatory waxes.

10. The system of claim 9, wherein said brush of said applicator has:
a) a handle; and
b) a plurality of bristles.

11. The system of claim 10, wherein said handle of said brush of said applicator has:
a) a proximal end;
b) a distal end; and
c) a midsection between said proximal end of said handle of said brush of said applicator and said distal end of said handle of said brush of said applicator.

12. The system of claim 11, wherein said plurality of bristles of said brush of said applicator extend axially from said proximal end of said handle of said brush of said applicator.

13. The system of claim 10, wherein said midsection of said handle of said brush of said applicator is arcuate so as to allow said brush of said applicator to rest safely across said rim of said vessel, between applications of said depilatory.

14. The system of claim 10, wherein said handle of said brush of said applicator is 5″ long.

15. The system of claim 10, wherein said handle of said brush of said applicator is made of plastic.

16. The system of claim 10, wherein said plurality of bristles of said brush are 1″ long.

17. The system of claim 10, wherein said plurality of bristles of said brush are synthetic.

18. The system of claim 10, wherein said handle of said brush of said applicator is 5″ long;
wherein said handle of said brush of said applicator is made of plastic;
wherein said plurality of bristles of said brush are 1″ long; and
wherein said plurality of bristles of said brush are synthetic.

19. A method of using a system for applying a depilatory to human skin using an applicator, wherein the applicator is a brush having a handle that is arcuate and a plurality of bristles extending axially from a proximal end of the handle of the brush of the applicator, and the depilatory includes bees wax, rosin, and natural plant-based oils, and wherein said method comprising the steps of:
a) melting the depilatory to a temperature of approximately 65° C. so as to have a consistency and color of warm honey or slightly thicker than latex paint and form a melted depilatory;
b) dipping the plurality of bristles of the brush of the applicator into the melted depilatory so as to form a plurality of wax-laden bristles of the brush of the applicator;
c) brushing the melted depilatory onto the skin in sections approximately 3 mm thick using the plurality of wax-laden bristles of the brush of the applicator so as to a form an applied depilatory;
d) allowing the applied depilatory to cool in approximately 15 seconds so as to form a strip of cooled depilatory; and
e) removing quickly the strip of cooled depilatory from the skin in one quick pull, up and away from a direction of hair growth.
